(19) United States
(12) Patent Application Publication
(10) Pub. No.: US 2017/0196666 A1
(43) Pub. Date: Jul. 13, 2017

(54) PRINTABLE AND SINTERABLE DENTAL COMPOSITIONS FOR PRODUCING PARTS OF DENTAL PROSTHESES AND METHOD FOR PRODUCING SAME

(71) Applicant: Heraeus Kulzer GmbH, Hanau (DE)

(72) Inventors: Uwe BÖHM, Hanau (DE); Mario BEYER, Bad Homburg (DE); Yvonne ZIEGLER, Hanau (DE); Jürgen STEIDL, Wöllstadt (DE)

(73) Assignee: Heraeus Kulzer GmbH, Hanau (DE)

(21) Appl. No.: 15/313,582

(22) PCT Filed: May 22, 2015
(86) PCT No.: PCT/EP2015/061409

§ 371 Date: Nov. 23, 2016

(30) Foreign Application Priority Data
May 23, 2014 (DE) 102014107330.4

(51) Int. Cl. A61C 13/00; B29C 67/00; B28B 11/24; B33Y 10/00; A61C 13/083; B33Y 80/00; A61K 6/00; A61K 6/02; A61K 6/027; A61K 6/083; B28B 1/00; B33Y 70/00

(52) U.S. Cl. CPC A61C 13/0019; B28B 1/001; B29C 67/0081; B28B 11/243; B33Y 10/00; B33Y 70/00; B33Y 80/00; A61K 6/0008; A61K 6/024; A61K 6/0273; A61K 6/083; A61C 13/083; A61C 13/0022; B29L 2031/7536

(57) ABSTRACT

Dental compositions and methods provide a flowable ceramic powder of a powdered component, wherein the compositions and methods comprise at least one dental glass, glass ceramics, metal oxide, mixed oxide selected from metal oxides and/or a mixture comprising at least two of said components, a printable dental ink. Moreover, the compositions and methods produce dental prosthetic form bodies.

The object of the invention is a dental composition of a flowable ceramic powder of a powdered component comprising at least one dental glass, glass ceramics, metal oxide, mixed oxide selected from metal oxides and/or a mixture comprising at least two of said components as well as a printable dental ink, a method for the production of dental prosthetic form bodies.

It is customary to produce dental ceramic prostheses from so-called blanks using a subtractive procedure. For this purpose, the prosthetic restorations, such as copings, crowns, and bridges, are milled out of the blankets and sintered in methods with specific firing curves. The known methods are associated with the disadvantage of their extensive consumption of materials. Moreover, very expensive 3- to 5-axes milling machines need to be procured for milling. For this reason, central milling centres have become established, which process the jobs from dental technicians and send the milled parts of prostheses back to the dental technician.

For the reasons specified above, there is a need for production procedures for dental prosthetic restorations working in additive manner. Moreover, a decentralised production of the prosthetic restorations is to be enabled.

It was the object of the invention to develop a method and compositions for the method enabling a production of dental prosthetic restorations without milling. Preferably, no more than some post-processing by polishing should take place. It was another object of the invention to produce prostheses that can be fit in the mouth of the patient without further adaptation of the dimensions of the prostheses. It was another object of the invention to provide a method that enables, at least in part, the use of pre-existing instruments and equipment of the dental technician, such as sintering furnaces. Moreover, a method was to be provided that enables the production of prostheses or parts of prostheses in decentralised manner.

The objects are met by a flowable composition of dental ceramic powders according to claim 1, a printable dental ink with a filling agent content of dental ceramic powdered components according to claim 8, as well as the method for printing dental form bodies that are suitable for the production of parts of dental prostheses or dental prostheses.

Presently, dental prostheses according to the invention shall be understood to mean all prosthetic restorations or parts of prosthetic restorations in the field of dentistry. Accordingly, dental prostheses or parts of dental prostheses according to the invention comprise both parts of removable dentures and parts of implant-born dentures. A dental prosthesis according to the invention shall also comprise dental prosthetic form bodies.

Surprisingly, it was feasible to provide a method for printing dental ceramics, in which contacting a dental ceramic powder, which is present as a powder bed, and a dental printable ink with an increased filling agent content allowed dental form bodies to be printed, in particular the foreign bodies can be printed layer-by-layer. The form bodies are printed by means of an additive method as blanks of a dental prosthetic restoration. The three-dimensional form bodies thus printed are dried under defined conditions and a green body is obtained. Subsequently, the binding agent is removed and the green body is sintered. A dental prosthetic restoration is obtained after the sintering.

The object of the invention is a dental composition of a flowable, in particular flowable, dental ceramic powder, in particular suited for the production of a powder bed for printing, whereby the powder contains a powdered component that comprises at least one dental glass, glass ceramics, metal oxide, mixed oxide selected from metal oxides and/or a mixture comprising at least two of said components, whereby the particle sizes, in particular the primary particles, of the powdered component are in the range of 2 nm to 200 μm, preferably of 2 nm to 150 μm, 1 nm to 100 μm, 1 nm to 15 μm. It is particularly preferable for the particle size d₉₀ to be less than or equal to 500 nm. Particle sizes of 2 nm to 100 μm, preferably of 2 nm to 50 μm, particularly preferably of 10 nm to 80 μm are also well-suited, preferably d₅₀ is less than or equal to 700 nm.

Preferably, the ceramic powder comprises, as total refractive index, a refractive index of 1.48 to 1.60 in the powdered components, in particular with a refractive index of 1.49 to 1.56, particularly preferably with a refractive index of 1.49 to 1.55, more preferably of 1.49 to 1.51, preferably 1.5 to 1.51.

According to the invention, the powdered component has a very narrow particle size distribution to prevent demixing during the transfer from the vibrating bulk cup and to enable the production of a homogeneous printing bed and/or powder bed. In this context, the particle size must not be too small since this can promote the formation of cracks in the printing bed.

Particularly preferably, the powdered component comprises the following particle size profile: d₅₀ less than or equal to 5.0 μm, in particular less than or equal to 1.0 μm, in particular less than or equal to 0.9 μm, preferably less than or equal to 0.7 μm, particularly preferably less than or equal to 0.6 μm, and, optionally, d₉₀=less than or equal to 11.0 μm, in particular less than or equal to 2.0 μm, in particular less than or equal to 1.8 μm, preferably less than or equal to 1.4 μm, particularly preferably less than or equal to 1.0 μm, and, optionally, d₉₉=preferably less than or equal to 12 μm, in particular less than or equal to 10 μm, in particular less than or equal to 5.0 μm, preferably less than or equal to 3.5 μm, preferably less than or equal to 2.0 μm. Said narrow particle size profile of the powdered components allows very homogeneous powder beds to be produced. Moreover, transparent dental prostheses were obtainable with said powdered component-containing inks and powder beds. Moreover, it is preferred for the inorganic pigments to comprise the above-mentioned particle size distribution profile.

The dental composition according to the invention of a pourable, in particular flowable, dental ceramic powder preferably comprises at least one excipient that allows the composition to be introduced into a printing bed without forming pores, inclusions, hollow spaces and/or clumps. The composition according to the invention therefore preferably has a particularly low angle of repose and preferably a particularly defined, preferably high, bulk density. For the reasons stated above, the flowability of the composition and the homogeneity have a direct influence on the quality of the green body and thus on the quality of the sintered form bodies as well. In customary processes for the production of translucent glass ceramic products, the glass products are formed in the molten state. Compositions according to the invention are produced using a 3D printing procedure without the application of external pressure and are subsequently dried, binding agent-depleted and sintered without further forming processes in order to obtain the final shape of the form body.

In particular dental glasses, dental ceramics, and metal oxides that can be used in the field of dentistry, which preferably meet the defined refractive index conditions and/or are used as radiopaquers, are considered as the dental powders, dental solids.

Dental glasses, glass ceramics, metal oxide, mixed oxide each with a refractive index of 1.48 to 1.60 in the powdered components, in particular with a refractive index of 1.49 to 1.56, particularly preferably with a refractive index of 1.49 to 1.55, more preferably of 1.49 to 1.51, preferably 1.5 to 1.51, are preferred. Preferably, other powdered components having a refractive index outside of said range are used only at a low content, in particular of less than or equal to 5% by weight in the total composition. For the production of transparent dental prostheses, the refractive index of all powdered components that are used is in a same narrow range, such as, preferably, of 1.49 to 1.51.

For the reasons stated above, it is necessary to produce a particularly homogeneous and dense powder bed for the printing process, whereby the powder bed, in addition, needs to be capable of being infiltrated by a filling agent-laden ink. Only compositions enabling the production of a powder bed of this type are well-suited for the ceramic 3D printing process.

Moreover, there are special refractive index requirements to be met by the material properties of the dental ceramic powders as well as the filling agents, the dental powdered components, of the ink and of the printing bed. Dental ceramic powders having a refractive index of 1.48 to 1.55 are particularly preferred.

Whereby the ceramic form body has a refractive index after sintering of 1.40 to 1.60, in particular of 1.49 to 1.56. Particularly preferred powdered components for the production of the form bodies, such as dental glasses or metal oxides, comprise a refractive index of 1.49+/−0.5. Powdered components according to the invention can be selected from dental glass ceramics, dental glasses, oxide ceramics, and metal oxides.

Particularly well-suited dental powdered components of the composition and ink, each independently comprise precipitated silicic acids, dental glasses such as aluminosilicate glasses or fluoroaluminosilicate glasses, bariumaluminium silicate, strontium silicate, strontium borosilicate, lithium silicate, lithium disilicate, lithiumaluminium silicate, layered silicates/phyllosilicates, calcium oxide, cerium oxide, potassium oxide, sodium oxide, borosilicates, borosilicate glasses, zeoliths, ytterbium-containing dental glasses, ytterbium fluoride-containing dental glasses, amorphous spherical fillers based on oxide or mixed oxide, in particular mixed oxides of SiO₂ and ZrO₂, glass fibres. Whereby the above-mentioned components can, in addition, comprise carbon fibres, as well as mixtures comprising said powdered components.

Dental powdered components comprising zirconium dioxide and ytterbium oxide or zirconium dioxide and ytterbium oxide and/or ytterbium fluoride are particularly preferred. Alternative preferred dental powdered components comprise mixtures of

a) silicon dioxide, mixed oxides of zirconium dioxide and silicon dioxide or precipitated silicic acids, and, optionally,

b) dental glasses such as aluminosilicate glasses or fluoroaluminosilicate glasses, barium-aluminium silicate, strontium silicate, strontium borosilicate, lithium silicate, lithium disilicate, lithiumaluminium silicate, borosilicates and/or borosilicate glasses, and, optionally,

c) calcium oxide, cerium oxide, potassium oxide, sodium oxide, and, optionally

d) ytterbium, ytterbium oxide, ytterbium fluoride, or a mixture comprising a) + b), or a), b) and c) or a) and c) or a) and d) or a), b), c) and d). Whereby the dental powdered components are used in the powder bed and/or in the ink.

The composition according to the invention and the filling agent-containing ink are used to produce three-dimensional blanks of parts of prostheses or entire prostheses from in an additive printing process. In this context, the shrinkage in the drying and sintering process is taken into consideration in the printing process and molded parts with appropriately larger dimensions are printed.

A preferred powdered component can comprise lithium disilicate glass ceramics, in particular of the following composition SiO₂ 57.0 to 80.0% by weight, Al₂O₃ 0 to 5.0% by weight, La₂O₃ 0.1 to 6.0% by weight, MgO 0 to 5.0% by weight, ZnO 0 to 8.0% by weight, Li₂O 11.0 to 19.1% by weight, with La₂O₃+La₂O₂ 0.1 to 7.0% by weight and MgO and ZnO 0.1 to 9.0% by weight. Moreover, the lithium disilicate can comprise ZrO₂ 0 to 10.0% by weight, K₂O 0 to 13.5% by weight, P₂O₅ 0 to 11.0% by weight, colouring and fluorescence components 0 to 8.0% by weight and additional components 0 to 6.0% by weight, ytterbium fluoride.

Moreover, a preferred dental powdered component can comprise, in % by weight, SiO₂ 57-80; Li₂O 11-19; K₂O 0.001-13; P₂O₅ 0.001-11; ZrO₂ 0.001-8; ZnO 0.001-8; other oxides and ceramic pigments 0-10.

Likewise preferred dental glasses comprise metal oxides such as SiO₂, Al₂O₃, B₂O₃, Na₂O, K₂O, Cs₂O, Na₂O+K₂O+Cs₂O, CaO, BaO, SrO, ZnO, ZrO₂, La₂O₃, Sc₂O₃, Y₂O₃, Nb₂O₅, Gd₂O₃, Yb₂O₃, Y₂O₃ Li₂O as well as P₂O₅, fluorine, YF₃, CerO₂, TiO₂, SnO₂, MgO, Sb₂O₃.

Likewise, the powdered components can comprise amorphous spherical filling agents, in particular as powdered component, in the dental ink, preferably based on oxide or mixed oxide, in particular mixed oxides of SiO₂ and ZrO₂, glass fibres. In general, it is preferred to use powders with spherical particles as powdered components. It is preferred to consider particles having an aspect ratio as defined below as being spherical. Particularly preferably, the aspect ratio of the particles is less than or equal to 1.4.

For the production of powder beds (synonymous to printing bed), it is preferred to use powdered components having a particle size d₉₀ of less than or equal to 100 micrometer (μm), preferably the particle sizes d₅₀ are less than or equal to 50 μm, in particular 0.001 to 50 μm, in particular less than or equal to 30 μm, more preferably less than or equal to 20 μm, and particularly preferably d₅₀ is less than or equal to 0.1 to 35 μm. Particularly preferably, the particle sizes d₉₀ are less than or equal to 2.5 μm, more preferably less than or equal to 1.5 μm. Moreover, d₅₀ preferably is less than or equal to 1.0 μm, preferably less than or equal to 0.8 μm. It is preferred according to the invention for d₉₀ to be 1.3 μm +/−0.5 μm, in particular +/−0.2 μm and d₅₀ to be 0.7 μm +/−0.5 μm, in particular +/−0.2 μm. It is particularly preferred to use powdered components having particle sizes d₉₉ of less than or equal to 480 nm, whereby particle sizes d₅₀ of less than or equal to 150 nm and d₉₉ of less than or equal to 460 nm are particularly preferred.

It is particularly preferred to use powdered components comprising zirconium dioxide, silicon dioxide, mixed oxides of zirconium dioxide and silicon dioxide, lithium disilicate having particle sizes d₉₉ of less than or equal to 480 nm, whereby particle sizes d₅₀ of less than or equal to 150 nm and d₉₉ of less than or equal to 460 nm are particularly preferred. Said particle sizes can preferably be used in the powder bed as at least one fraction and/or in the dental ink. In this context, it is preferred to use said powdered components having particle sizes d₉₉ of less than or equal to 480 nm, whereby particle sizes d₅₀ of less than or equal to 150 nm and d₉₉ of less than or equal to 460 nm are particularly preferred, and, optionally, at a weight ratio of 0.01 : 50 : 1 to dental powdered components of particle sizes d₅₀ of less than or equal to 5.0 μm, in particular less than or equal to 1.0 μm, in particular less than or equal to 0.9 μm, preferably less than or equal to 0.7 μm, particularly preferably less than or equal to 0.6 μm and more than or equal to 500 nm, and, optionally, d₉₀ of less than or equal to 11.0 μm, in particular less than or equal to 2.0 μm, in particular less than or equal to 1.8 μm, preferably less than or equal to 1.4 μm, particularly preferably less than or equal to 1.0 μm and, each independently, more than or equal to 500 nm, and, optionally, d₉₉ is preferably less than or equal to 12 μm, in particular less than or equal to10 μm, in particular less than or equal to 5.0 μm, preferably less than or equal to 3.5 μm, preferably less than or equal to 2.0 μm and, each independently, more than or equal to 500 nm.

It has been evident that the best results concerning transparency and/or translucence can be obtained in the printing process when the dental compositions have a bulk density of more than or equal to 0.4 g/cm³, in particular more than or equal to 0.75 g/cm³, particularly preferably more than or equal to 0.80 g/cm³. Preferably, the dental compositions comprise an angle of repose of less than or equal to 60°, in particular less than 55°, preferably less than 50°, more preferably less than or equal to 45°, in particular in combination with the bulk density specified above.

Moreover, pigments can be added to the dental composition in order to adjust the basic colouring of the prostheses or parts of prostheses, which can be adapted further in colour according to need by means of the pigmentation of the inks. Preferably, the pigments comprise a similar particle size as the powdered components such that the homogeneity of the powder bed is not impaired. In particular, no demixing shall take place when the powder is transferred from a vibrating bulk cup. However, pigments usually comprise particle sizes d₉₀ of less than or equal to 20 μm to less than 15 μm and also less than 10 μm. Customarily, d₅₀ is approx. 0.001 to 10 μm. It can therefore be of advantage to formulate the pigments for production of the powder bed in order to adapt their particle size to that of the powdered components. This is feasible by formulating them together with the powdered component and/or with binding agents, for example in a granulation.

Compositions that are present in the form of a powder bed as well as the use of the compositions for the production of powder beds for 3D printing processes are also objects of the invention. In particular, the compositions are present as planar powder bed, particularly preferably with a) a bulk density of more than or equal to 0.45 g/cm³, in particular with a bulk density of more than or equal to 0.5 g/cm³, in particular more than or equal to 0.80 g/cm³ to 1.2 g/cm³, more preferably more than or equal to 0.85 g/cm³, more preferably more than or equal to 0.90 g/cm³, 0.95 g/cm³ and/or b) with a tamped density of more than or equal to 2.0 g/cm³, in particular more than or equal to 3 g/cm³, more preferably more than or equal to 4.0 g/cm³ (DIN 51916), preferably more than or equal to 5.0 g/cm³, more than or equal to 6.0 g/cm³, more than or equal to 7.0 g/cm³, more than or equal to 8.0 g/cm³. Dental compositions that are present compacted as a powder bed for printing are a object of the invention.

Preferably, the flowable composition contains at least one excipient comprising dispersing agents, flow additives, peptising agents, binding agents, excipients for regulating the surface tension of solids, lubricants, additives against the build-up of static charge (antistatic agents). It is particularly preferred to use excipients of pharmaceutical quality in the compositions, preferably those that are suitable for the production of powders, since pharmaceutical powders usually have a particle size of less than 100 micrometres. Antistatic agents can be selected, for example, from cationic surfactants, such as quaternary ammonium compounds.

Typical inorganic lubricants for the production of powders having sufficient spreadability or pourability can be talcum, zinc oxide, white clay (Bolus alba), titanium dioxide, magnesium oxide, magnesium carbonate, silicon, and highly disperse silicon dioxide, such as Aerosil 200 (refractive index n=1.452). Depending on the translucent properties, some can be used preferably in the composition. Stearates, starch, modified starch, and lactose are conceivable as organic lubricants.

Known flow additives comprise precipitated silicic acids. Conceivable binding agents for the production of the flowable composition include binding agents that are preferably solid and non-tacky at room temperature and do not impair the bulk properties of the composition, but rather improve them. Alternatively, a binding agent can be formulated onto the powdered components, if applicable together with further excipients such as flow additives, by means of spray drying. Possible solid binding agents comprise polyvinyl alcohol, sugar, polyacrylic acids, celluloses such as the cellulose ethers specified below or, just as well, TEOS.

Another object of the invention is a composition that contains at least one excipient, whereby the excipient, in particular, comprises flow additives (silicon dioxide, in particular having a primary particle size of less than or equal to 100 nm, such as Sipernat, an Aerosil, precipitated silicic acid comprising primary particles of less than or equal to 20 nm, binding agents, such as gum arabic, rubber, cellulose ethers such as methylcellulose, ethylhydroxylethylcellulose (low surface tension), hydroxypropylcellulose, PEG, gelatin, alginate, starch, tragacanth, sugar, saccharides, dextrin, bentonites, in particular iron-free bentonites (platelet size: 5-10 nm in thickness, 0.1-2 μm in length), polyvinylpyrrolidone, polyvinylalcohol, possibly resins, polyacrylic acids or polyacrylates, urethane, whereby the excipients preferably are of a pharmaceutical quality with an impurity content of less than 1% by weight.

According to a further embodiment, a object of the invention is a printable liquid dental ink, in particular a printable composition, comprising
at least one dental powdered component (also called filling agent), comprising at least one dental glass, glass ceramics, metal oxide, mixed oxide selected from metal oxides and/or a mixture comprising at least two of the specified components, whereby the refractive indices of the powdered components preferably are in the range of 1.48 to 1.60 and, in particular, the powdered component is at least partially silanised and/or functionalised with metal acid esters; and
at least one liquid component and, optionally,
at least one excipient, in particular the at least one excipient specifically comprising dispersing agents, in particular one dispersing agent, polymeric dispersing agent, flow additives, peptising agents such as alkaline and/or alkaline earth salt of carboxylic acids, such as citrates, tartrates, oxalates or bisphosphonates, silanolates; binding agents, antistatic agents and/or excipient for regulation of the surface tension.

According to a particularly preferred alternative of the invention, the powdered components are silanised and preferably the ink is adjusted to a pH value of less than 8, preferably less than 5, particularly preferably less than 4. A particularly preferred ink comprises zirconium dioxide as powdered component and, optionally, further powdered components, whereby it is further preferred to have the zirconium dioxide and, optionally, the further components be silanised or be treated with a metal ester, whereby it is further preferred to have the pH value of the ink adjusted to a pH of less than or equal to 8.

According to a further alternative, the powdered components, preferably zirconium oxides, are provided with an organic coupling reagent. Suitable coupling reagents comprise metal acid esters, such as zirconiumaluminates of the following structure

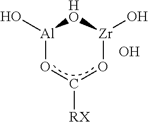

whereby X═—NH₂, —COOH, —COO′, hydroxyphenyl-, methacryl-, carboxyphenyl-, alkyl-, mercapto-, phenyl-, —H, vinyl-, styryl-, melamin-, epoxy- or aryl- and R═(CH₂)ₙ—, whereby n=0 to 12. Zirconiumaluminates based on the following carboxylic acids are particularly well-suited: HOOC—(CH2)4—COOH; HOOC—(CH2)4—NH2, olefinic carboxylic acid, X being alkyl with 1 to 12 C-atoms is particularly preferred.

A filling agent-laden printable liquid dental ink comprising at least one inorganic pigment as well as, preferably, 1 to 30% by weight of dental powdered component is particularly preferred. It is preferred to use at least one inorganic coloured pigment or a mixture of inorganic coloured pigments.

Customary inorganic pigments can be used as inorganic pigments. Suitable pigments for the production of dental inks must be temperature-resistant during the firing process. Accordingly, inorganic pigments that are essentially colour-stable up to about 850° C., preferably up to 1,200° C., are well-suited. Accordingly, one object of the invention is a dental ink comprising at least one inorganic pigment possessing colour stability above 850° C., in particular from 850 to 1,050° C., preferably from 900 to 930° C. The selected pigment is selected according to the chemical composition of the ceramic powders and components of the matrix.

Customary inorganic pigments essentially comprise oxides, oxide hydrates, sulfides, sulfates, carbonates, and silicates of the transition metals such as, preferably, pigments comprising zirconium and/or iron. Known pigments comprise zirconium silicates, iron silicates, iron oxides, manganese oxides, chromium oxides, γ-Fe₂O₃, Fe₃O₄/Fe₂O₃, Cr₂O₃, TiO₂. Moreover, the inorganic pigments can comprise coloured pigments, luster pigments, metal effect pigments, in particular luster pigments with platelet- or scale-shaped metal particles, such as iron oxide red, strontium yellow, aluminium bronze or silver bronze, pearlescent pigments or gold bronze, aluminium bronze.

Particularly preferred inorganic coloured pigments comprise iron oxides, zirconium-praseodymium silicate (Zr,Pr)SiO₄ (CAS no.: 68187-15-5), zirconium-iron silicate (Zr, Fe(SiO₄)), (CAS no: 68412-79-3), zinc-iron-chromium-brown-spinel (Zn,Fe)(Fe,Cr)₂O₄, (CAS no.: 68186-88-9), chromium-tin-pink-sphene CaO:SnO₂SiO₂:Cr₂O₃, (CAS no.: 68187-12-2).

The liquid component can be selected from compounds that are liquid at room temperature (liquid). Water, water-miscible solvents such as alcohols, ethanol, isopropanol, polyols, ester, ketone, ether, ethyl ester are preferred. Due to the subsequent sintering process and the desire to minimise shrinkage, it is preferred to use liquids as liquid component that can be removed as early as in the drying process with virtually no residue and low volume shrinkage.

The inks according to the invention preferably contain at least one excipient, whereby the excipient comprises flow additives such as silicon dioxide, in particular highly-disperse silicon dioxide having a primary particle size of less than or equal to 100 nm, such as Sipernat, Aerosil, precipitated silicic acid comprising primary particles of less than or equal to 20 nm, liquefying agents, wetting agents, binding agents, such as gum arabic, rubber, cellulose ethers such as methylcellulose, ethylhydroxylethylcellulose (low surface tension), hydroxypropylcellulose, PEG, gelatin, alginate, starch, tragacanth, sugar, saccharides, dextrin, bentonites, in particular iron-free bentonites (platelet size: 5-10 nm in thickness, 0.1-2 μm in length), polyvinylpyrrolidone, polyvinylalcohol, possibly resins, epoxy resins, urethane, polyacrylic acids, whereby the excipients preferably comprise an impurity content of less than 1% by weight. The above-mentioned peptising agents can comprise alkaline and/or alkaline earth salt of carboxylic acids, such as citrates, tartrates, oxalates, tin(II) chloride as well as bisphosphonates, silanolates. The particle sizes of the excipients present in both the flowable composition and the powdered component of the ink are preferably in the same range as the powdered components as disclosed above.

Inks that are particularly preferred according to the invention comprise the following individual properties or combinations of properties:

a) a viscosity in the range of 1 to 50 mPa·s, in particular 5 to 30 mPa·s, preferably 10 to 25 mPa·s, particularly preferably of 0.1 to 15 mPa·s , 5 to 15 mPa·s, and/or

b) a surface tension of less than or equal to 50 mN/m, 35.1 mN/m, less than 40 mN/m, 35 mN/m, 30 mN/m, particularly preferably less than or equal to 20 mN/m, such as from 0.01 to 50 mN/m, and/or c) a content of powdered components (solids content) from 0.01 to 50% by weight, in particular from 1 to 30% by weight, 2% by weight to 30% by weight, in particular 0.01 to 5% by weight, such as approximately 1, 2, 3, 4, 5, 6, 7, 8, 9, 10, 11, 12, 13, 14, 15% by weight, preferably approximately 10% by weight. Particularly preferred powdered components are powdered components whose refractive index corresponds to that of the powdered components of the composition for production of the printing bed. It is preferred to strive for a high solids content or filling agent content in the ink in order to minimise the shrinkage during sintering. At the same time, the powdered component in the ink is to impart no opacity to the sintered form body. In general, highly translucent form bodies and prostheses are desired. Preferably, the ink comprises properties a, b, and c, particularly preferably with 5 to 30 mPa·s, less than or equal to 40 mN/m, in particular less than or equal to 30 mN/m, and a powdered component content ranging from 1 to 30% by weight. A particularly preferred content of powdered component in the dental ink ranges from 1 to 35% by weight, particularly preferably from 5 to 35% by weight, 10 to 25% by weight, alternatively preferably 12 to 35% by weight.

It also preferred for the ink to have a surface tension of less than or equal to 50 mN/m, preferably the surface tension is less than or equal to 40 mN/m, less than or equal to 30 mN/m, preferably less than 20 mN/m.

Also preferably, the ink comprises, each independently, at least one pigment for producing coloured inks by means of which defined colour profiles of the prosthetic parts can be printed in the printing process.

Moreover, an object of the invention is an ink that comprises
1 to 50% by weight powdered components, in particular an at least partially silanised powdered component,
1 to 90% by weight liquid component, whereby the liquid component, in turn, comprises
   (a) 1 to 90% by weight water, in particular 15 to 60% by weight, preferably 15 to 55% by weight, and
   b) 0 to 70% by weight of at least one water-miscible organic diluting agent, whereby a) and b) together can account for 1 to 90% by weight of the ink. Preferred water-miscible diluting agents b) comprise b.1) monohydroxy-functional alcohols, such as ethanol, isopropanol, and, optionally, b.2) as second component liquid di- and/or tri- to multifunctional alcohols and/or polyethers such as dioles, trioles, in particular ethylene glycol, glycerol, PEG, whereby the sum b) of components b.1) and b.2) comprises from 0 to 70% by weight
   b.1) 0 to 70% by weight monohydroxy alcohol, in particular 5 to 40% by weight, preferably 10 to 30% by weight,
   b.2) 0 to 70% by weight diol, triol, multivalent alcohol, in particular 5 to 40% by weight, preferably 5 to 35% by weight, whereby the total composition of the liquid component is 100% by weight, and
0.001 to 10% by weight of at least one excipient, such as a dispersing agent, preferably 0.001 to 5% by weight, more preferably 0.01 to 2% by weight, whereby the total composition is present at 100% by weight, whereby the total composition of the ink adds up to 100% by weight.

Further water-miscible diluting agents and/or organic solvents can comprise ester, ketone, ether, ethyl ester, butyl ester. And further solvents known to a person skilled in the art. It is preferred to use polar organic solvents, such as a aprotic or protic polar solvents.

Moreover, the ink preferably comprises at least one pigment, preferably one inorganic pigment, whereby 0 to 5% by weight, in particular 0.001 to 5% by weight, preferably 0.01 to 2% by weight of the at least one pigment is/are present in the ink, whereby the total composition adds up to 100% by weight.

The preferred particle sizes of the powdered component in the ink are preferably markedly smaller than that of the composition in order to enable good infiltration of the ink into the printing bed. Therefore, the particle sizes of the powdered components in the ink are preferably less than or equal to 25 μm, in particular less than or equal to 20 μm, particularly preferably less than or equal to 5 μm, in particular d₅₀ less than or equal to 4.5 μm, particularly preferably less than or equal to 2 μm, in particular d₉₀ less than or equal to 2 μm, preferably d₅₀ less than or equal to 1 μm. The particles preferably are spherical in shape.

The pH value of the powdered component in the ink preferably is between 1 and 9. According to the invention, the pH value of the ink is being adjusted to a pH of less than or equal to 5, particularly preferably to a pH of less than or equal to 4. The adjustment can be done, for example, by adding quaternary ammonium compounds.

Another object of the invention is a dental ink comprising coloured and/or white pigments or mixtures of pigments, whereby the pigments, in particular, have a particle size d₉₀ of less than or equal to 20 μm, preferably less than 15 μm, particularly preferably less than or equal to 10 μm, more preferably d₅₀ is approximately 0.001 to 10 μm, particularly preferably, the ink comprises the above-mentioned inorganic pigments as pigments. According to the invention, the pigments comprise a particle size profile in accordance with the disclosure on page 3 regarding the powdered component.

A dental ink that can be distributed in droplets with a diameter of 10 to 100 μm, in particular up to a droplet size of 40 to 80 μm, particularly preferably from 40 to 70 μm, 40 to 60 μm, preferably approximately 50 μm +/−5 μm is also preferred. In this context, it is preferable for the ink to be printable over an extended period of time, such as 30 minutes, preferably 1 to 24 hours, further preferably up to 1 week, at an essentially constant droplet size.

Another object of the invention is a dental ink from which droplets with an aspect ratio of diameter 1 to diameter 2 of 0.2 to 5, in particular less than or equal to 2 to more than or equal to 0.5, in particular from 1.4 to 0.7 are generated, whereby diameter 1 and diameter 2 are arranged at an angle of approximately 90° with respect to each other. Particularly preferably, approximately spherical droplets can be generated from the ink. Preferred droplet sizes are described above, whereby it is particularly preferred to be able to generate droplets of less than or equal to 20 μm, preferably less than or equal to 10 μm, in particular less than or equal to 5 μm through the selection of a suitable printing head.

Dental inks, whose droplets remain approximately spherical after exit from the printing nozzle, are particularly preferred Preferably, the droplets remain approximately spherical until they hit the powder bed. Preferably with the aspect ratio defined above, in particular with an aspect ratio of 1.4 to 0.7.

The production of the printable dental ink involves the components being mixed with each other and, preferably, being post-treated with ultrasound, in particular in order to convert aggregates into individual particles preferably sized 10 to 200 nm, and to homogenise the composition. Any aggregation of powdered particles is to be prevented.

Another object of the invention is a dental ink that comprises an organic diluting agent in the liquid component, such as mono-hydroxy-functional alcohols or ketones, such as acetone (b.1), and/or at least one anti-drying additive, such as, each independently, at least one polyether, polyethylene glycol, polypropylene glycol, EO/PO polyether, whereby the polyether is preferred to have a molecular weight Mw of 100 to 6,000 g/mol, preferably 150 to 3,000 g/mol. Particularly preferred anti-drying agents comprise ethylene glycol and/or glycerin. Ethylene glycol and/or glycerin are particularly preferred.

In this context, the anti-drying agent is classified to be component b.2) and preferably is present at a content of 0.001 to 50% by weight, in particular 0.001 to 30% by weight, particularly preferably of 5 to 35% by weight.

According to a further alternative of the invention, a object of the invention is a method for printing three-dimensional form bodies that are preferably suitable for the production at least of parts of dental prostheses, as well as three-dimensional form bodies that can be obtained according to said method, whereby the method comprises process section (A), including the steps of:

(i) contacting a dental ink, in particular a dispersion, comprising at least one liquid component and at least one powdered component, in particular at least one inorganic particulate dental solid, to

(ii) at least a part of a layer, in particular a planar layer, of a composition comprising a dental ceramic powder of a powdered component comprising at least one dental glass, glass ceramics, metal oxide, mixed oxide selected from metal oxides and/or a mixture comprising at least two of said components, with the powdered components preferably comprising at least one excipient, such as flow additive and/or binding agent,

(iii) whereby the contacting causes the dental ceramic powder comprising the powdered component to be solidified at least at the point of contact at least in said layer of the dental ceramic powder or in the region of the point of contact, in particular of the powdered component,

(iv) applying another layer of the dental ceramic powder comprising the powdered component in particular onto layer (ii) and/or (iii), and

(v) performing steps (i), (ii), (iii), and (iv) at least once, preferably (v) repeatedly performing steps (i), (ii), (iii), and (iv).

According to the invention, steps (i) to (v) are performed for an appropriate number of times such that at least one moulded part of a blank of a prosthetic moulded part is formed in the powder bed, preferably a plurality of moulded parts of blanks are formed. Preferably fins can be provided at the moulded parts in order to minimise the shrinkage of the form bodies during sintering. Printed moulded parts, which may still contain binding agent, are considered to be blanks of a prosthetic moulded part. Green bodies can be produced from said blanks by removing the binding agent.

The powdered components in steps (i) and (iii) and (iv) are preferably present as powder bed, printing bed or are formed into a powder bed.

Another object of the invention is a method, in which a three-dimensional form body is obtained after process section (A) repeatedly after performing steps (i) to (v), in particular a dental three-dimensional form body that corresponds, at least in part, to a blank of a part of a dental prosthetic restoration.

Another object of the invention is a powder bed comprising form bodies of prosthetic parts that are obtainable according to the method according to the invention, in particular of med-tech parts, preferably of dental moulded parts. The form bodies are suitable for production of the green bodies.

Accordingly, an object of the invention is a printed form body comprising a dental ceramic powder in the form of a blank of a dental prosthesis, of a part of a dental prosthesis, of a part of a hoof, of a medical prosthesis, of a veneering, crown, bridge, inlay, onlay, veneer, superstructure, substructure, implant post, abutment, spacer, fin, a bone replacement such as part of a jaw bone or other bones. Another object of the invention is a printing of a ceramic framework about the form body such that the form body can remain connected via fins to a framework during the sintering.

Another object of the invention is a method comprising a process section (B) comprising the steps of (I) forming a green body from the form body, preferably from the blank, in particular by removing at least one volatile compound, preferably by drying at more than 100° C., in particular at approximately more than or equal to 110° C., preferably for a drying time of 1 to 36 hours, usually 24 hours, and (II) exposing the green body.

The exposing of the green body can take place by removing the non-solidified powder bed;

compressed air of variable strength can additionally be used for this purpose. Alternatively or additionally, the powder bed can be made to vibrate in order to remove loose powder bed.

Particularly preferably, the method can be performed in mono- to multicolour printing procedures, such as three-colour printing, in order to produce dental prostheses that are particularly pleasing aesthetically and mimic the colour profile and the aesthetics of natural teeth particularly well.

Another object of the invention is a method comprising a process section (C) comprising the steps of (I) optionally, removing the binding agent from the green body, (II) sintering the green body, (Ill) obtaining a three-dimensional dental form body that corresponds to at least a part of a dental prosthetic restoration. Particularly preferably, the sintered form body only needs to be after-worked superficially, for example by polishing, a colour application is to be done, such as by painting, and, if applicable, the bite is to be corrected.

A removal of the binding agents, also called binding agent removal, takes place at 500° C. before the sintering, in particular with a heating rate of up to 50 K/h, in particular of 50 K/h, whereby the temperature is maintained for approximately 5 minutes to 4 hours, in particular for 30 minutes. Cooling to room temperature proceeds rapidly, preferably at up to 100 K/h.

The (II) sintering, in particular of glass ceramics such as silicate compositions, preferably proceeds at a heating rate of up to 30 K/h to 700 to 1,200° C., in particular to 850 to 1,050° C., preferably to 850 to 950° C., possibly followed by cooling to room temperature. Preferably, the sintering at a temperature above 700° C., preferably above 850° C., takes place for between 10 seconds and 2 hours, preferably the sintering takes place for approximately 40 seconds to 2 minutes.

The (II) sintering, in particular of zirconium dioxide (ZrO₂) compositions, preferably takes place at a heating rate of 30 K/h, or with 300 to 600° C/h up to 700 to 1,800° C., in particular up to 1,450 or 1,500° C., preferably to 1,450 to 1,500° C., possibly followed by cooling to room temperature. Preferably, the sintering at a temperature above 1,000° C., preferably above 1,350° C., takes place for between 10 seconds and 2 hours, preferably the sintering takes place for approximately 40 seconds to 2 minutes.

The (II) sintering, in particular of aluminium oxide (Al₂O₃) compositions, preferably takes place at a heating rate of 30 K/h, or from 300 to 600° C/h to 700 to 1,900° C., in particular up to 1,450 or 1,530° C., preferably to 1,450 to 1,530° C., possibly followed by cooling to room temperature. Preferably, the sintering at a temperature above 1,100° C., preferably above 1,400° C., takes place for between 10 seconds and 2 hours, preferably the sintering takes place for approximately 40 seconds to 2 minutes.

Another object of the method is the application of at least one layer of a dental ceramic powder having a layer thickness of 0.1 μm to 100 μm; in particular a layer thickness of 1 to 30 μm, more preferably of 1 to 25 μm, 5 to 20 μm, particularly preferably of 5 to 15 μm, preferably of 10 μm (+/−5 μm (micrometre), +/−2 μm) is applied. According to the invention, a multitude of layers having the above-mentioned layer thicknesses are applied, in particular in step (iv). An object of the invention is a method comprising process sections (A), (B), and (C).

Another object of the invention is a method comprising a step with the following firing curve, whereby the firing curve optionally has a first temperature phase (binding agent removal) in the range of 150 to 500° C., in particular ranging from 1 to 48 h, preferably from 1 to 24 h, and a second temperature phase (sintering) in the range of 550° C. to 1,100° C., in particular of 830° C. to 1,100° C., preferably of 880° C. to 1,100° C., preferably approximately 900° C. to 1,050° C. +/−10° C., whereby the temperature preferably is increased at a rate of 5° C/minute, in particular 10° C/minute, preferably 20° C/minute, particularly preferably 30° C/minute, until the second phase is reached. The temperature of the second phase is maintained preferably for more than or equal to 1 minute, up to 60 minutes, in particular between 0.25 and 5 minutes, preferably approximately 1 to 2 minutes.

Depending on the powdered components, a temperature of 860° can be too low for the production of a translucent sample; in these cases, the temperature can be increased incrementally up to 1,250° C. in order to find the temperature for the production of translucent samples that is specific for the respective dental material.

Another object of the invention is a method comprising step (iv) of applying a layer of the flowable ceramic powder, in particular of the powdered component, by dispensing the ceramic powder from an oscillating bulk cup with at least one opening, preferably an elongated opening provided on the bottom side, onto the existing layer or over an overflow edge.

Preferably, a vibrating bulk cup (funnel) is used for application of the dental composition of the flowable ceramic powder; preferably, the bulk cup vibrates at a defined frequency in order to apply a homogeneous layer of the powdered component. Moreover, it is preferred for the bulk cup to be assigned to a rotating vibrator. Moreover, it is preferred for the bulk cup to be assigned to softer rubber buffers.

Adapted thereto, a device, in particular a doctor blade, a roller, antistatic roller is rolled over the applied layer in order to homogenise and, optionally, compact, the layer.

Preferably, the oscillating bulk cup is operated at a frequency of maximally 450 Hz, preferably less than or equal to 300 Hz, preferably between 100 and 300 Hz. The rate, at which the bulk cup is advanced over the layer, preferably is between 10 mm/s and 500 mm/s, preferably between 50 and 400 mm/s, preferably approximately 200 mm/s +/−20 mm/s.

Another object of the invention is a method comprising the step, in which, after application of the porable, in particular flowable dental ceramic powder, in particular of the powdered component, from the bulk cup or during the application of the dental ceramic powder from the bulk cup, a device compacts the dental ceramic powder and forms it into a layer, in particular into a planar layer for printing, which preferably can be infiltrated by droplets of the dental ink.

The device can comprise a doctor blade, a roller, whereby the roller preferably comprises a diameter of 10 to 30 mm, preferably 10 to 25 mm, approximately 20 mm or approximately 15 mm, 18 mm, 10 mm.

Another object of the invention is a software for the production of dental prosthetic restorations by means of which a defined leading signal can be set in addition to the printing pattern per layer in the printing process. A preferred leading signal can be set to approximately −1.375 mm in order to place the droplets in the powder bed exactly at the coordinates corresponding to the CAD data. The aim is not to have any droplet offset of superimposed z-data with identical x,y data in the printing bed. However, the exact leading signal depends on the viscosity of the ink, the design of the printer and on the printing head and is therefore to be set individually. Moreover, the software for fabrication of prosthetic restorations can be used to set the droplet size, the line distance and/or the droplet distance just in freely selectable manner. Accordingly, the software settings can be used to ensure that the droplets contact the powder bed exactly at the position corresponding to the CAD data set. The accuracy of the printing head that is used in this context is approximately: (x-, y-, z-axis): +/−3 μm, the accuracy of the height setting of the printing platform is +/−5 μm and the accuracy of the layer thickness is 25 μm, 15 μm, preferably 10 μm. The software can modify the CAD data sets of prosthetic moulded parts for print processing.

According to a further alternative, one object of the invention is a kit comprising starting materials for three-dimensional printing at least of parts of ceramic prostheses, such as dental prostheses, or for printing of dental prostheses, whereby the kit comprises, as separated components, (A) a dental composition of a flowable dental ceramic powder of the type described above, in particular for production of a powder bed, and, as component (B), a printable liquid dental ink of the type described above.

Another object of the invention is a dental three-dimensional green body obtainable by the method according to the invention, in particular a pigmented green body obtainable by the method according to the invention.

A part of a dental prosthesis or a dental prosthesis obtainable according to the method according to the invention is also an object of the invention. Particularly preferably, a pigmented prosthesis can be obtained according to the method, more preferably a pigmented, translucent prosthesis, preferably a dental prosthesis, part of a dental prosthesis, can be obtained according to the method. The prostheses can be further adapted through customary after-treatments, such as grinding, polishing, colouring. Preferably, only the sintering fins are removed from the prostheses and the prostheses can then be fitted-in in the patient.

According to the invention, the translucency of the at least one part of the dental prosthesis or of a dental prosthesis, such as veneer, front tooth, veneering, in particular a prosthesis for full-anatomical purposes, obtainable by the method according to the invention is more than or equal to 5% [relative translucency], in particular more than or equal to 8 to 20%, particularly preferably from 10 to 18% [relative translucency]. The translucency was measured by means of direct light transmission using a digital photo-radiometer in a dark chamber (light source: 150 W halogen lamp). Alternatively, the translucency can be measured with a SF-600 Datacolor.

Another object of the invention is the use of a three-dimensional printer for printing powdered dental ceramic compositions. Another object of the invention is the use of a three-dimensional printer for printing printable dental inks for the production of blanks, in particular by means of the combination of the printer, the powdered dental compositions and the printable dental ink for the production of at least parts of dental prosthetic restorations, in particular of dental prostheses, bridges, crowns, veneers, artificial teeth, dental veneers, implants, implant posts, abutments, dental spacers, superstructures, substructures, telescopic crowns, onlays and/or inlays, or med-tech prostheses, such as parts of limbs, parts of hooves, hooves, bone parts, vertebral bodies, parts of facial bone.

The use of an oscillating bulk cup with at least one bottom-side opening, preferably with a bottom-side elongated opening for application of at least one layer of a flowable ceramic powder, in particular of the powdered component, onto the existing layer or whereby the application takes place via an overflow edge, is particularly preferred. Another object of the invention is the use of a roller in the printer that serves to make planar the further layer of the ceramic powder applied from the oscillating bulk cup onto the existing layer of ceramic powder in a plane and, in particular, in order to homogenise and, optionally, compact the layer.

The use of three-dimensional printers for printing veneerings, dental copings, crowns, bridges, implant posts, abutments, spacers, whereby ZPrinter® printers such as Z 150, Z 250, Z350, Z450, Z650 as well as specially adapted modifications of the printers or any other 3D printer can be used as preferred printers, is particularly preferred. It is preferred to use printing heads in the printer that have openings for dispensation of the printable compositions, whereby the openings preferably have a diameter of 50 to 200 μm, preferably of 50 to 150 μm, preferably approximately 100 μm. It is particularly preferred to use printing heads with heatable nozzle. Another object of the invention is the use of a three-dimensional printer for printing ceramic dental compositions.

The invention is illustrated in more detail based on the following examples without limiting the invention to the examples given.

### EXAMPLES

### Examples 1 to 8—Ink

Ink: The inks were prepared by mixing components (i), (ii), and (iii) with each other, with the sum adding up to 100% by weight. In order to prepare homogeneous dispersions, the samples were after-treated in an ultrasonic bath.

(i) Filling agent in ink:

a) Zirconium dioxide stabilised with 3 mol-% Y₂O₃; 15 wt.-% (Example 1)

b) Zirconium dioxide stabilised with 3 mol-% Y₂O₃; 20 wt.-% (Example 2)

c) Zirconium dioxide stabilised with 3 mol-% Y₂O₃; 25 wt.-% (Example 3)

d) Zirconium dioxide stabilised with 3 mol-% Y₂O₃; 30 wt.-% (Example 4)

e) Zirconium dioxide stabilised with 3 mol-% Y₂O₃; 35 wt.-% (Example 5)

f) Zirconium dioxide stabilised with 3 mol-% Y₂O₃; 40 wt.-% (Example 6)

g) Zirconium dioxide stabilised with 3 mol-% Y₂O₃; 45 wt.-% (Example 7)

h) Zirconium dioxide stabilised with 3 mol-% Y₂O₃; 5 wt.-% (Example 8)

Particle sizes: a) approx. 0.7 μm, d₉₀=1.3 μm, d₉₉<1.9 μm

b) to f) d₅₀ less than or equal to 500 nm, d₉₀ less than or equal to 1.0 μm

g) d₅₀=560 nm, d₉₀ less than or equal to 1.0 μm

Viscosity of samples a) to g) from 40 to 15 mPas

(ii) liquid component:

Water: 20 to 55 wt.-%

Ethanol 10 to 30 wt.-%

Ethylene glycol: 5 to 40 wt.-%

(iii) Excipient dispersing agent/liquefying agent: 0.2 wt.-% Dolapix CE 64.

### Examples 9 and 10—Ink

TABLE 1a
Composition Ink

| In wt.-% | Ex. 9 (A3) | Ex. 10 | Ex. 8 | Ex. 6a | Ex. 6b | Ex. 11 |
|---|---|---|---|---|---|---|
| Ethanol | 24.0 | 14.0 | 35.0 | 13.0 | 4.5 | 62.3 |
| Ethylene glycol | 31.8 | 7.8 | 25.0 | Glycerol; | 9.0 | |
| | | | | 22.0 | | |
| Water (dist.) | 24.0 | 49.0 | 34.5 | 24.5 | 42 | |
| Filling agent | 20.0 (SiO₂ | 29.0 | 5 (h, | 40 (ZrO₂, 3 | 40 wt.-% | 37.5 |
| | raw material, | (Aerosil | ZrO₂, 3 | mol-% | (ZrO₂, 3 | (ZrO₂, 3 |
| | see Table 2, | Ox50) | mol-% | Y₂O₃) | mol-% | mol-% |
| | 4, A3) | | Y₂O₃) | | Y₂O₃), | Y₂O₃) |
| Dispersing agent | 0.2 Dolapix | 0.2 | 0.5 | 3.48 | 0.4 Dolapix | 0.2 |
| | CE 64 | | | | CE64 | |
| | | | | | 9.0 Byketol-PC | |
| Viscosity | | 19.28 mPa · s | 13.4 mPa · s | 3.48 mPa · s | | 3.25 mPa · s |
| Surface tension | | 35.1 mN/m | 55.1 mN/m | 48.3 | | 47.7 |

TABLE 1b
Compositions Ink

| | | Example 13, | |
|---|---|---|---|
| In wt.-% | Example 12 | A3 | Example 14 (A4) |
| Ethanol | 35.0 | 94.5 | 24.0 |
| Ethylene | | | 31.8 |
| glycol | | | |
| Water (dist.) | | | 24.0 |
| Filling agent | 60 (ZrO₂, 3 mol-% | 5.0 (SiO₂ raw | 20.0 (SiO₂ raw |
| | Y₂O₃)¹ | material, A3) | material, see Table |
| | | | 2, 4, A4) |
| Dispersing | 5.0 | 0.5 | 0.2 Dolapix CE 64 |
| agent | | | |
| Viscosity | 6.48 | 13.4 | |
| Surface | 35.1 | 55.1 | |
| tension | | | |

¹Particle size distribution as in examples 1 to 8

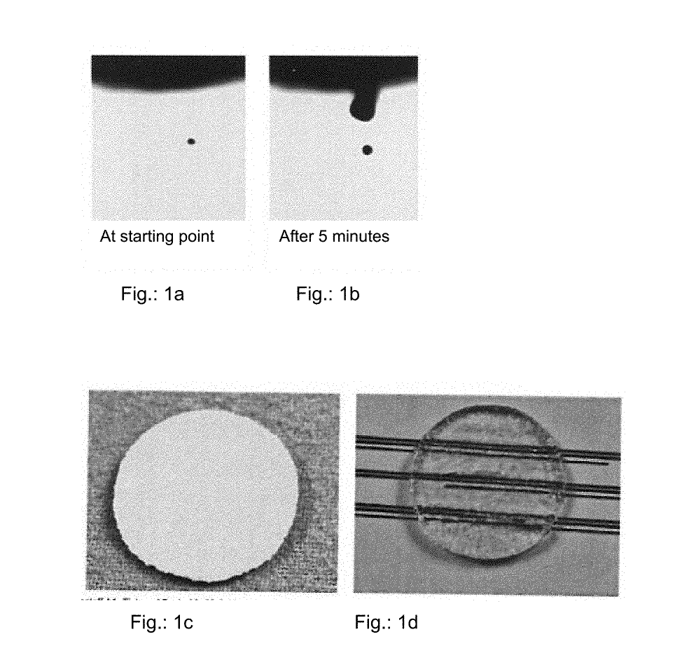

The ink and the droplets showed sufficient stability during the printing process (FIG. 1a and b; droplet settings: Voltage/pulse length 58/28, frequency 500 Hz after approx. 5 min., droplet size approx. 48 μm). Dental ceramic filling agent in the ink (powdered component): d₅₀=4.6 μm, d₁₀₀=17 μm

TABLE 2
Filling agent in wt.-% in dental ink and powder bed - (composition
material SiO₂ raw material: A3, A2, A1)

SiO₂ 70.45%, Al₂O₃ 11.80%, K₂O 8.50 5, Na₂O 5.0%, Li₂O 1.5%, CaO
1.0 5, B₂O₃ 0.30%, F 0.6%, CeO₂ 0.85%

Powder bed (Table 3): flowable dental ceramic powder zirconium dioxide stabilised with 3 mol-% Y₂O₃

TABLE 3
Powder bed samples 1 and 2 dental ceramic powder - zirconium dioxide

| | Samples | |
|---|---|---|
| | 1 | 2 - silanised |
| Composition | ZrO₂, 3 mol-% Y₂O₃ | ZrO₂, 3 mol-% Y₂O₃ |
| d₅₀ | 0.7 μm | 0.7 μm |
| Bulk density: [g/cm³] | 3.61 | 3.33 |
| Angle of repose [°] | 35.3 | 34.3 |
| Theoretical density: | 6.02 | |
| [g/cm³] | | |
| Packing density [%] | 60 | 55 |

Powder bed (Table 4): flowable dental powder SiO₂ raw material—raw material Table 2

TABLE 4
Powder bed samples A1, A2, A3, and A4

| | Powder | | | | |
|---|---|---|---|---|---|
| | A1 (SiO₂ | | | | |
| | raw | | | | |
| | material, | | | | |
| | Table 2) | A2 | A3 | A4 | A3 |
| Composition | | | +7% PVA | | Spray |
| | | | | | drying |
| d₁₀ | | | <0.3 μm | <1.83 μm | <0.2 μm |
| d₅₀ | <5.0 | <0.5 μm | <0.7 μm | <0.56 μm | <0.6 μm |
| d₉₀ | <10.8 | <1.0 μm | <1.3 μm | <1.03 μm | <1.7 μm |
| d₉₉ | | <2.0 μm | <1.84 μm | | |
| BET [m²/g] | 1.28 | | | | |
| Bulk density | 0.42 | | | | |
| [g/cm₃] | | | | | |
| Angle of repose | 52 | | | | |
| [°] | | | | | |

Samples printed with a 3D printer. Microdrop MD-K-140-020 100 μm, <20 mPas,

Printer: Servo drive: x/y: max: 600 mm/s, z max: 0.1 μm, axial resolution: 0.1 μm, positioning accuracy +/−3 μm.

Lifting floor: process speed: max. 50 mm/s, internal axial resolution: 0.1 μm, positioning accuracy: +/−5 μm,

Installation space: 50×50×50 mm

Printing resolution: 25 μm

Droplet size: 50-60 μm

Positioning accuracy: +/−3 μm

Bulk cup: Travel rate: max. 200 mm/s

Vibration frequency: max. 300 Hz

Layer thickness: 25 μm

Example 1 Ceramics: The line distance, the droplet distance and the interference between lines are being defined. Powder bed: Sample A1, ink, example 9 (A3)

Cylindrical form bodies are being printed. Drying time Production Green body: 110° C., 24 h. Binding agent removal: t/T curve: Heating 50K/h to 500° C., hold time 30 minutes, Cooling: 100 K/h to room temperature

Sintering: a) Heating: 30 K/h to 869° C., hold time 1 minute (from 300° C. in vacuum), Cooling: within 15 minutes

Transparent dense form bodies of the above-mentioned samples were obtained. b) Powder bed A (Table 4) and ink example 9 (A3) were printed to form a form body and the binding agent was removed at 550° C. (and the sintering process took place at 850° C., 50 K/h). Transparent samples according to FIG. 1d were obtained. FIG. 1c shows the form body before the sintering process.

The addition of inorganic pigments, such as zirconium-praseodymium silicate (Zr,Pr)SiO4. CAS no.: 68187-15-5, zirconium-iron silicate (Zr, Fe(SiO4), (CAS no: 68412-79-3) with d₅₀<5 μm and the use of a printing head with four ink containers allowed coloured translucent form bodies to be produced.

Using the ink according to example 9 and adding pigments, a form body of a crown was printed (shrinkage calculated) and sintered. A crown with translucent properties was obtained after the sintering process.

Example 2: Powder bed: Sample A3+PVA (Table 4), ink example 9. The procedure was analogous to example 1. Translucent and transparent form bodies were obtained. No inclusions of air were observed.

Example 3: Powder bed: Sample A1, ink, example 10 The procedure was analogous to example 1.

Example 4: Powder bed: Sample A3+PVA, ink example 10. The procedure was analogous to example 1.

Example 5: An ink with added pigment and solid (example 9 (A3), 0.5 wt.-% pigment 3240 yellow) and powder bed sample A3+PVA analogous to example 1 was printed and sintered.

Example 6: An ink with added pigment and solid (example 9 (A3), 0.5 wt.-% pigment 3240 yellow) and powder bed sample A2 analogous to example 1 was printed and sintered.

Example 7: An ink with added pigment and solid (example 9 (A3), 0.5 wt.-% pigment PS 3210 (pink)) and powder bed sample A3+PVA analogous to example 1 was printed and sintered.

Example 8: Powder bed: Sample A3+PVA, ink example 13. The procedure was analogous to example 1. Translucent and transparent form bodies were obtained. No inclusions of air were observed.

Example 9: Powder bed: Sample A3+PVA, ink example 14. The procedure was analogous to example 1. Translucent and transparent form bodies were obtained. No inclusions of air were observed.

Example 10 Ceramics: Powder bed: Table 3 Sample 1, ink example 8 The procedure was analogous to example 1. Translucent and transparent form bodies were obtained. No inclusions of air were observed.

Example 11 Ceramics: Powder bed: Table 3 Sample 1, ink example 6b. The procedure was analogous to example 1. Translucent and transparent form bodies were obtained. No inclusions of air were observed.

Example 12 Ceramics: Powder bed: Table 3 Sample 1, ink example 12 The procedure was analogous to example 1. Translucent and transparent form bodies were obtained. No inclusions of air were observed.

Example 13 Ceramics: Powder bed: Table 3 Sample 2, ink example 8 The procedure was analogous to example 1. Translucent and transparent form bodies were obtained. No inclusions of air were observed.

Example 14 Ceramics: Powder bed: Table 4 Sample A3, ink example 9 (A3). The procedure was analogous to example 1. Translucent and transparent form bodies were obtained. No inclusions of air were observed.

Example 15 Ceramics: Powder bed: Table 3 Sample 1 at a weight ratio of 1 : 1 to sample 1* with d₉₉ being less than or equal to 500 nm, ink example 3. The procedure was analogous to example 1. Translucent and transparent form bodies were obtained. No inclusions of air were observed.

Example 16 Ceramics: Powder bed: Table 3 Sample 1 at a weight ratio of 1 : 1 to sample 1* with d₉₉ being less than or equal to 150 nm, ink example 5. The procedure was analogous to example 1. Translucent and transparent form bodies were obtained. No inclusions of air were observed (more than 15% [rel. translucency]).

1. A dental Dental composition of a flowable dental ceramic powder suitable for the production of a powder bed for printing, wherein the flowable dental powder contains a powdered component comprising at least one component selected from one dental glass, glass ceramics, metal oxide, mixed oxide selected from metal oxides and a mixture comprising at least two of said components wherein particle sizes of the powdered component are in the range of 2 nm to 200 μm.

2. The dental composition according to claim 1, wherein the powdered component comprises at least one component selected from zirconium dioxide, silicon dioxide, mixed oxides of zirconium dioxide and silicon dioxide, precipitated silicic acids, dental aluminosilicate or fluoroaluminosilicate glasses, bariumaluminium silicate, strontium silicate, strontium borosilicate, lithium silicate, lithium disilicate, lithiumaluminium silicate, layered silicates/phyllosilicates, calcium oxide, cerium oxide, potassium oxide, sodium oxide, ytterbium, ytterbium oxide, ytterbium fluoride, borosilicates, borosilicate glasses, zeoliths and a mixture comprising at least one of said components.

3. The dental composition according to claim 1, wherein the powdered components has a particle size of d₉₀ of less than or equal to 100 micrometre (μm).

4. The dental composition according to claim 1, wherein the composition has a bulk density of more than or equal to 0.4 g/cm³.

5. The dental composition according to claim 1, wherein the composition has an angle of repose of less than or equal to 60°.

6. The dental composition according to claim 1, wherein the composition is present as a powder bed.

7. The dental composition according to claim 1, wherein the composition contains at least one excipient comprising at least one selected from flow additives, lubricants, antistatic agents, dispersing agents and binding agents.

8. A printable liquid dental ink, comprising
at least one dental powdered component comprising at least component selected from one dental glass, glass ceramics, metal oxide, mixed oxide selected from metal oxides and a mixture comprising at least two of said components,
at least one liquid component, and, optionally,
at least one excipient selected from a dispersing agent, an anti-drying additive, a flow additive, a peptising agent, a binding agent and an excipient for regulation of the surface tension.

9. The dental ink according to claim 8 wherein the ink has
a) a viscosity of 1 to 50 mPa·s, and/or
b) a surface tension of less than or equal to 50 mN/m and/or
c) the content of powdered components is 0.01 to 50% by weight.

10. The dental ink according to claim 8, wherein the ink comprises
1 to 50% by weight powdered components
1 to 90% by weight liquid component, whereby the liquid component comprises
a) 1 to 90% by weight water, and
b) 0 to 70% by weight of at least one water-miscible organic diluting agent in the total composition of the liquid component, and
0.001 to 10% by weight of at least one excipient, such as dispersing agent, whereby the total composition of the ink adds up to 100% by weight.

11. The dental Dental ink according to claim 8, wherein the powdered component has particle sizes of less than or equal to 5 μm.

12. The dental ink according to claim 8, wherein the ink inorganic coloured pigments.

13. The dental ink according to claim 8, wherein the ink is distributable in droplets with a diameter of 10 to 100 μm.

14. The dental ink according to claim 8, wherein the liquid component contains at least one organic diluting agent and/or comprises at least one anti-drying additive such as polyether, polyethylene glycol, polypropylene glycol, EO/PO polyether, ethylene glycol and/or glycin.

15. The dental ink according to claim 13, wherein the droplets can be generated from the ink that have an aspect ratio of diameter 1 to diameter 2 of 0.2 to 5, whereby diameter 1 and diameter 2 are arranged at an angle of approximately 90° with respect to each other.

16. A method of printing three-dimensional form bodies that are suitable for the production at least of parts of dental prostheses, the method comprising the process section (A), including:
(i) contacting a dental ink comprising at least one liquid component and at least one dental powdered component, to
(ii) at least a part of a layer of a dental ceramic powder,
(iii) whereby the contacting causes the dental ceramic powder to be solidified at least at the point of contact at least in said layer of the powder or in the region of the point of contact,
(iv) applying another layer of a flowable dental ceramic powder, in particular onto the layer (ii) and
(v) performing (i), (ii), (iii), and (iv) at least once.

17. The method according to claim 16, wherein, after process section (A) after repeatedly performing (i) to (v) at least one dental three-dimensional form body is obtainable that corresponds, at least in part, to a blank of a part of a dental prosthetic restoration.

18. The method according to claim 17, wherein the method further comprises a process section (B) including:
(I) forming a green body from the form body, and
(II) exposing the green body.

19. The method according to claim 18, wherein the method further comprises a process section (C) including:
(I) optionally, removing the binding agent from the green body,
(II) sintering the green body, and
(III) obtaining a three-dimensional dental form body that corresponds to at least a part of a dental prosthetic restoration.

20. The method according to claim 16, wherein at least one layer of a dental ceramic powder having a layer thickness of 0.1 μm to 100 μm is being applied or present.

21. The method according to claim 16, wherein (iv) the applying of a layer of the flowable ceramic powder takes place by dispensing the ceramic powder from an oscillating bulk cup with an elongated opening provided on the bottom side or over an overflow edge onto the existing layer.

22. The method according to claim 16, wherein, after application of the flowable dental ceramic powder from the bulk cup or during the application of the dental ceramic powder from the bulk cup, a device compacts the dental ceramic powder and forms it into a layer.

23. A kit comprising starting materials for three-dimensional printing at least of parts of dental prostheses, whereby the kit comprises, as separated components, (A) a dental composition of the flowable dental ceramic powder according to claim 1 and, as component (B), a printable liquid dental ink.

24. A powder bed comprising form bodies of prosthetic parts that obtainable according to the method according to claim 16.

25. A dental three-dimensional green body obtainable according to the method according to claim 16.

26. A port of a dental prosthesis or dental prosthesis obtainable according to the method according to claim 16.

27. (canceled)

28. (canceled)
